(19) United States
(12) Patent Application Publication
(10) Pub. No.: US 2021/0402167 A1
(43) Pub. Date: Dec. 30, 2021

(54) OCCLUSION APPARATUS FOR INFLOW CONDUIT

(71) Applicant: Evaheart, Inc., Houston, TX (US)

(72) Inventor: Tadashi MOTOMURA, Houston, TX (US)

(21) Appl. No.: 17/363,308

(22) Filed: Jun. 30, 2021

Related U.S. Application Data
(63) Continuation of application No. PCT/US2019/068953, filed on Dec. 30, 2019.
(60) Provisional application No. 62/787,058, filed on Dec. 31, 2018.

(51) Int. Cl. A61M 39/20; A61M 60/178; A61M 60/863; A61B 17/00

(52) U.S. Cl. CPC A61M 39/20; A61M 60/178; A61B 2017/00676; A61B 17/0057; A61M 60/863

(57) ABSTRACT

An apparatus for occluding the inflow conduit of a ventricular assist device comprising a plug body, a plug surface, and a plug cap. The plug cap is configured to extend around an external surface of the cylindrical body of the plug body. The plug cap may be removeably connected to the plug body.

This application is a Continuation of International Application No. PCT/US2019/068953, filed on Dec. 30, 2019, which claims the benefit of priority to U.S. Provisional Application Ser. No. 62/787,058, filed on Dec. 31, 2018, which applications are incorporated herein by reference. To the extent appropriate, a claim of priority is made to each of the above-disclosed applications.

### BACKGROUND

Treatment of cardiomyopathy often requires the implantation of a left ventricular assist device (LVAD). In a small percentage of patients, the LVAD may be removed when the patient regains normal heart function. A standard explantation procedure includes mobilization of the LVAD, removal of the sewing ring, and closure of the hole in the myocardium of the apex. However, this often leads to surgical trauma, myocardial injury, blood clot formation, brain stroke, bleeding, the loss of myocardium tissue, and distortion of the left ventricle geometry.

Traditionally, when the LVAD is removed from a patient who regains normal heart function, the entire LVAD is removed. The inflow conduit and the suture cuff of the LVAD is removed from the left ventricular apex. The removal procedure is often conducted under cardiopulmonary bypass and the cored left ventricular apex is closed with a surgical procedure. The surgical repair of the left ventricular apex causes blood loss and distortion of the left ventricular geometry, which leads to surgical mortality and impaired reserved heart function.

To mitigate these problems, other solutions remove the inflow conduit of the LVAD and replace it with a titanium plug. The titanium plug is inserted into the suture cuff to fill the empty left ventricle apex defect. This procedure still requires cardiopulmonary bypass, and the protruding plug remains inside the left ventricular chamber. The patient may be predisposed to blood clot formation, which again may cause several embolic complications, such as a stroke.

Other solutions include anchoring a low profile titanium plug to the apical suture cuff using surgical sutures, applying titanium-sintered beads to the plug surface to enhance tissue ingrowth, and using a hemostatic sealant to prevent blood leakage from the gap between the plug and the suture cuff. The low profile plug may not align with the apical endocardium plane due to variability of myocardium thickness. A mismatch between myocardial thickness and plug height may cause blood flow disturbance and stagnation, which may lead to blood clot formation. However, these solutions require a cardiopulmonary bypass.

Therefore, there is a need for an improved device and method of closing the hole in the myocardium of the apex during an explantation of the LVAD procedure that simplifies the procedure and does not require a cardiopulmonary bypass.

### SUMMARY

This invention is generally related to a cardiovascular medical product, namely, an occlusion apparatus for placement within an inflow conduit of an LVAD. Aspects of the method include providing a device that can be implanted without the need for a cardiopulmonary bypass.

In a first aspect, an apparatus for occluding an inflow conduit of a ventricular assist device comprises a plug body, a plug surface, and a plug cap. The plug body has a superior end and an inferior end and a cylindrical body extending from the superior end to the inferior end. The plug surface is located at the superior end of the plug body. The plug surface covers the cylindrical body and has a first side and a second side. The plug cap is configured to extend around an external surface of the cylindrical body of the plug body.

In another aspect, a method for occluding a hole in the apex of the heart following an explantation procedure of a ventricular assist device is described. The method includes inserting an occlusion apparatus into an inflow conduit of the VAD. The occlusion apparatus comprises a plug body having a superior end and an inferior end and a cylindrical body extending from the superior end to the inferior end, and a plug surface at the superior end of the plug body, the plug surface covers the cylindrical body and has a first side and a second side, and a plug cap is located at the inferior end of the plug body.

In yet another aspect, a means for occluding an inflow conduit of a ventricular assist device is described. The means for occluding includes a means for filling the volume of the inflow conduit and a means for securing. The means for securing is connected to the means for filling the volume of the conduit.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

The following drawings are illustrative of particular embodiments of the present disclosure and therefore do not limit the scope of the present disclosure. The drawings are not to scale and are intended for use in conjunction with the explanations in the following Detailed Description. Embodiments of the present disclosure will hereinafter be described in conjunction with the appended drawings, wherein like numerals denote like elements.

FIG. 1 illustrates an exploded view of an example embodiment of an occlusion apparatus.

FIG. 2 illustrates an example embodiment of an occlusion apparatus.

FIGS. 3A-3D illustrate an example implantation procedure of an occlusion apparatus.

FIGS. 4A-4C illustrate another example embodiment of an occlusion apparatus.

FIGS. 5A-5C illustrate an alternative embodiment of an occlusion apparatus.

FIG. 6A illustrates a cross-sectional view of a plug body.

FIG. 6B illustrates a cross-sectional view of a plug cap.

FIGS. 7A-7C illustrate alternative embodiments of an occlusion apparatus.

### DETAILED DESCRIPTION

The figures and descriptions provided herein may have been simplified to illustrate aspects that are relevant for a clear understanding of the herein described devices, systems, and methods, while eliminating, for the purpose of clarity, other aspects that may be found in typical devices, systems, and methods. Those of ordinary skill may recognize that other elements and/or operations may be desirable and/or necessary to implement the devices, systems, and methods described herein. Because such elements and operations are well known in the art, and because they do not facilitate a better understanding of the present disclosure, a discussion of such elements and operations may not be provided herein. However, the present disclosure is deemed to inherently include all such elements, variations, and modifications to the described aspects that would be known to those of ordinary skill in the art.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

Embodiments of the present invention, for example, are described above with reference to block diagrams and/or operational illustrations of methods, systems, and computer program products according to embodiments of the invention. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

Continuous flow left ventricular assist device (cf-LVAD) is a lifesaving medical device to support the heart function for end-stage heart failure. Contrary to current application of cf-LVAD (bridge to heart transplantation or destination therapy), there is a potential cardiac recovery in 4-8 percent of ventricular assist device (VAD) patients, who eventually regain normal cardiac function and do not require further VAD support. Those patients eventually require removal (explantation) of the cf-LVAD pump and inflow conduit.

LVAD (or VAD) systems comprise an inflow conduit that is placed within the apex of the left ventricle. The inflow conduit is inserted through a hole created in the apex of the left ventricle. VADs also include a pump, batteries, and tubing. Generally, the pump is connected to the inflow conduit and the tubing extends from the pump and back to the aorta.

The inflow conduit is placed through a hole created in the apex of the heart, generally the left ventricle. The inflow conduit is a drainage pipe that can remain in the apex even after an explantation procedure of a VAD with the use of an occlusion apparatus as described in detail below. The occlusion apparatus solves the problem stated above because there is no need to remove the inflow conduit of the VAD, which enables the elimination of a cardiopulmonary bypass. Further, using the occlusion apparatus as described herein allows for the anatomy of the apex to remain intact. Still further, the occlusion apparatus as described herein allows the inflow conduit to remain within the heart in the chance that the patient needs a VAD to be re-implanted at a later time.

The occlusion apparatus avoids bleeding backflow from the left ventricle through the inflow cuff. Further, the dead space within the inflow cuff is filled after implantation to avoid blood clots. Due to filling of the plug, the plug has minimum flow disturbance in the left ventricle apex. The occlusion apparatus also includes multiple channel grooves on an exterior surface for air removal. The plug also includes a textured surface on the top of the plug where the blood is exposed at the apex, which prevents blood clotting. The cap also includes multiple side holes for air removal. Alternatively, a space or gap exists between the plug and the inner lumen of the inflow conduit. This allows for air removal via an air removal pathway.

The occlusion apparatus as described herein does not require a cardiopulmonary bypass for implantation, and does not interfere with intraventricular blood flow around the left ventricular apex, which can prevent blood clots.

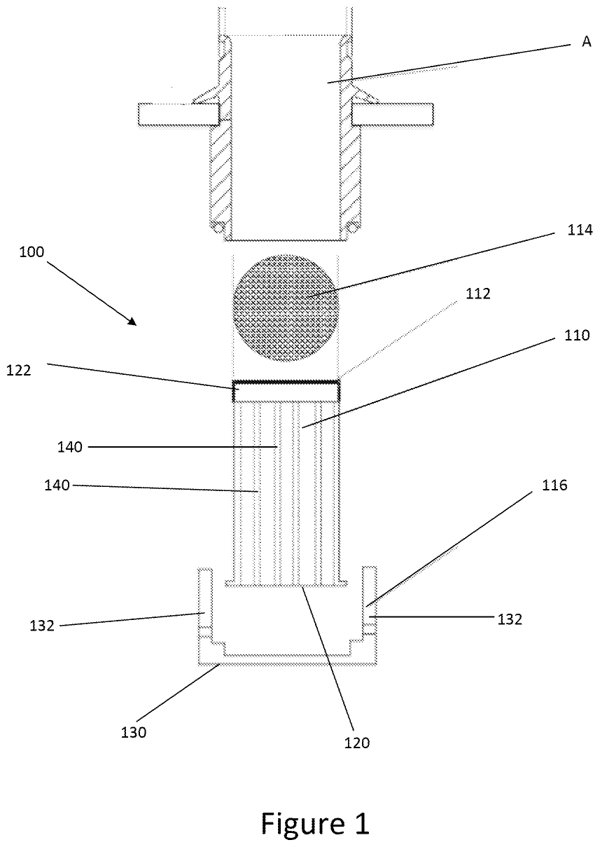

FIG.1 illustrates an example embodiment of an occlusion apparatus 100. The occlusion apparatus 100 is designed to be placed in the inflow conduit A of a VAD. The inflow conduit A of a VAD creates a lumen in the apex of the heart, which allows blood to flow from the heart through a mechanical pump and back to the body. The inflow conduit A may be the inflow conduit of any VAD known in the art.

The occlusion apparatus 100 includes at least a plug body 110, a plug tip 112 comprising a superior plug surface 114, and a plug cap 116. The plug body 110 is cylindrical in shape with a superior end 122 and an inferior end 120. The plug body 110 has a constant diameter, the diameter size selected to fit within inflow conduit A. In a first embodiment, the plug body 110 is hollow, while in a second embodiment, the plug body 110 is solid or semi-solid.

A plug tip 112 is located on the superior end 122 of the plug body 110. The plug tip 112 includes a plug surface 114, which is made from a material that encourages tissue ingrowth.

The plug body 110 also includes an inferior end 120, which may or may not be open.

The plug body 110 includes a plurality of grooves 140 that extend in a longitudinal direction along the outside of the plug body 110. The plurality of grooves 140 provide a pathway for blood to flow during air removal when the occlusion apparatus 100 is implanted within the inflow conduit A. The plurality of grooves 140 may also allow for air removal in the space between the plug body 110 and the inflow conduit A.

In an embodiment, the plug body 110 includes 4-12 grooves. In an example embodiment, the plug body 110 has eight grooves that are equidistantly spaced around the exterior surface. The grooves 140 may be from about 1 mm to about 4 mm deep and from about 1 mm to about 4 mm wide. In an embodiment, the plug body 110 includes grooves 140 that are about 2 mm deep. In an embodiment, the plug body 110 includes grooves 140 that are about 2 mm wide. The grooves 140 function to create a channel between the left ventricle and the exterior of the heart for easy air removal during implantation.

The plug body 110 is sized to fit completely within the inflow conduit A of any traditional VAD. In an example embodiment, the plug body 110 completely fills the inflow conduit A to prevent pooling of blood between an internal surface of the inflow conduit A and the external surface of the plug body 110. Preventing the pooling of blood reduces the chance of blood clots, and also helps to prevent brain injury.

In another embodiment, the plug body 110 does not completely fill the interior space of the inflow conduit A. In an embodiment wherein there is space between the interior of the inflow conduit A and the plug body 110, the plug body 110 may be expandable to fill the space within the inflow conduit A. For example, the plug body may have an exterior diameter of from about 14 mm to about 18 mm.

In an exemplary embodiment, the plug body 110 is made from materials that do not change shape or are rigid. For example, the plug body 110 may be made from biocompatible materials, such as stainless steel, titanium, alumina, Nitinol, or polymers. Alternatively, the plug body 110 may be made from a material that is malleable and/or expandable, and able to fill the space of the inflow conduit A, such as a biocompatible silicone or polyurethane. A malleable plug body 110 is able to be inserted through a curved or bent inflow conduit.

The occlusion apparatus 100 also includes a plug surface 114 located at a superior end of the plug tip 112. The plug surface 114 extends across the plug tip 112 to cover the area of the plug tip 112 that is exposed to blood within the heart. In an embodiment, the plug surface 114 includes a single layer, while in another embodiment, the plug surface 114 includes multiple layers. In an embodiment having a single layer, the plug surface 114 has a textured surface to encourage tissue ingrowth. In an embodiment comprising multiple layers, the plug surface 114 comprises an inferior layer comprising a biocompatible material, and a superior layer comprising a material encouraging tissue ingrowth.

Materials encouraging tissue ingrowth may be polytetrafluoroethylene (PTFE), polyesters, or cell seeding fabrics. Example fabrics are textile surgical meshes, or other woven fabrics, such as Debakey double valor fabric. The plug surface 114 may also be made from biocompatible materials, such as stainless steel, titanium, alumina, Nitinol, or polymers.

Still further, the superior surface of the plug surface 114 can be textured. A textured plug surface 114 may include a plurality of secured titanium beads that form a smooth, but textured surface. Alternatively, the plug surface 114 may include a rough textured surface. A textured surface helps to encourage tissue ingrowth. Still further, the superior surface of the plug surface 114 may be smooth and flat.

The occlusion apparatus 100 also includes a cap 116 located at an inferior end 120 of the plug body 110, and opposite the end of the plug tip 112. The cap 116 securely connects to the plug body 110 to prevent the flow of blood through the inflow conduit A. In an embodiment, the cap 116 is removeably connected to the plug body 110. A removable connection may be a screw-type, snap-fit, or other similar connection type.

The cap 116 includes an inferior surface 130 that is sized to cover the diameter of the plug body 110 and inflow conduit A. The cap 116 also includes a wall 132 that extends upward from the inferior surface 130. The wall 132 is located around an outside edge of the inferior surface 130 and is sized to extend around the exterior surface of the plug body 110. In an embodiment, within the inferior surface 130 of the cap 116, and along the wall 132, is an O-ring sealing mechanism. The O-ring sealing mechanism (not shown) prevents blood leakage. Example materials for the O-ring include biocompatible rubber and/or plastics. Alternatively, a hemostatic sealant may be used to prevent blood leakage.

The cap 116 also includes release apertures (not shown). The release apertures allow for the removal of air when the cap 116 is being connected to the plug body 110. The apertures are sized to allow air to escape. For example, the release apertures may be about 1 mm in diameter. Alternatively, the release apertures may be less than 1 mm in diameter, for example, 0.5 mm or 0.75 mm in diameter. Still further, the release apertures may be greater than 1 mm in diameter, for example, 1.25 mm or 1.5 mm in diameter. The apertures are placed every 30°-60°, for example, every 45° around the plug cap 116.

In an embodiment where there is a space or gap between the plug body 110 and cap 116, the space is from about 50 μm to about 2000 μm long. In such an embodiment, no release apertures are needed in the cap 116, as the air can be removed from the space or gap instead.

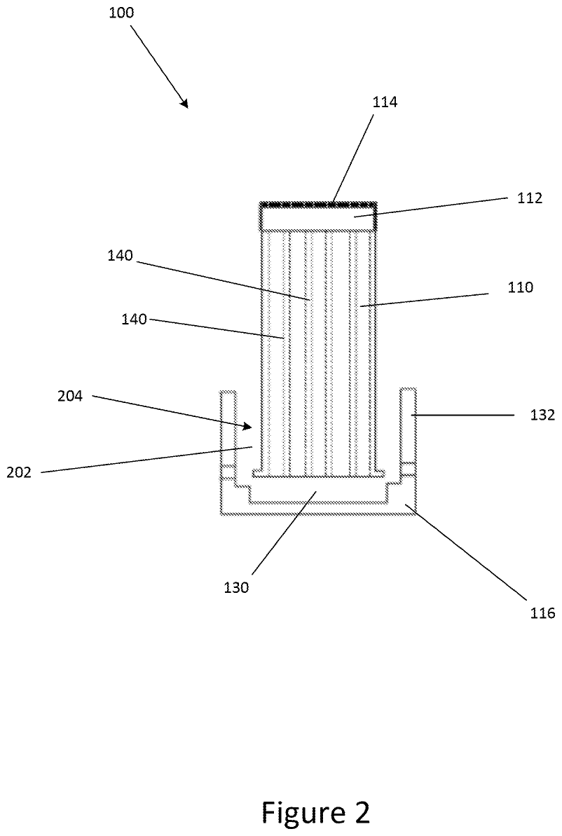

FIG. 2 illustrates a simplified side view of an example embodiment of an occlusion apparatus 100 in a state of being assembled. As shown (in cross-section), the cap 116 is placed in close contact with the plug body 110, so it may be connected to the plug body 110 to be completely assembled.

The cap 116 is sized to fit around the external diameter of the plug body 110, as well as the inflow conduit A (not shown) of the LVAD. Therefore, the cap 116 may include an additional space 202 between the walls 132 of the cap 116 and the plug body 110 that is sized to accommodate the width of the inflow conduit A.

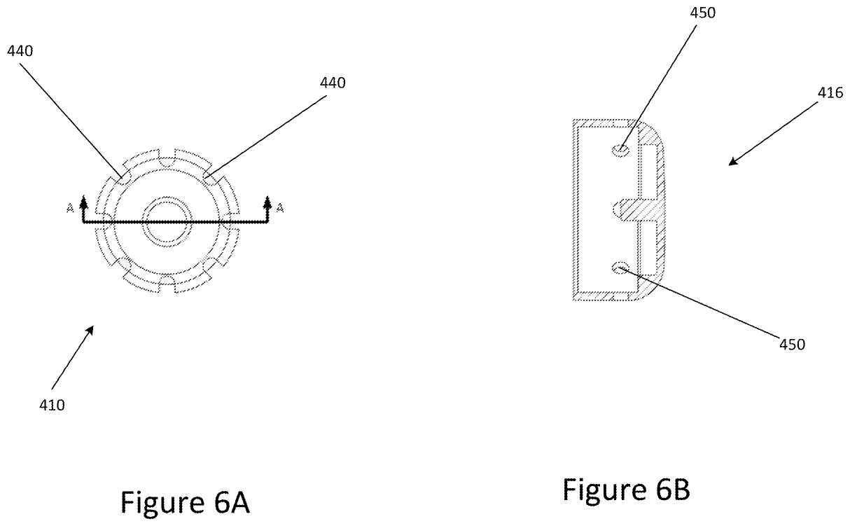

As shown, the cap 116 includes a recess 204, which is sized to accept the plug body 110 and the inflow conduit A. The wall 132 extends up from the inferior surface 130 to form the recess. For example, the wall 132 may be about 6 mm to about 12 mm tall. In an embodiment, the wall 132 is 9 mm tall. The wall 132 of the cap 116 is shown in more detail at FIG. 6B.

Also shown is the plug surface 114 adhered to the plug tip 112. As described above, the plug surface 114 is made from a biocompatible material as the plug surface 114 is exposed to blood within the heart.

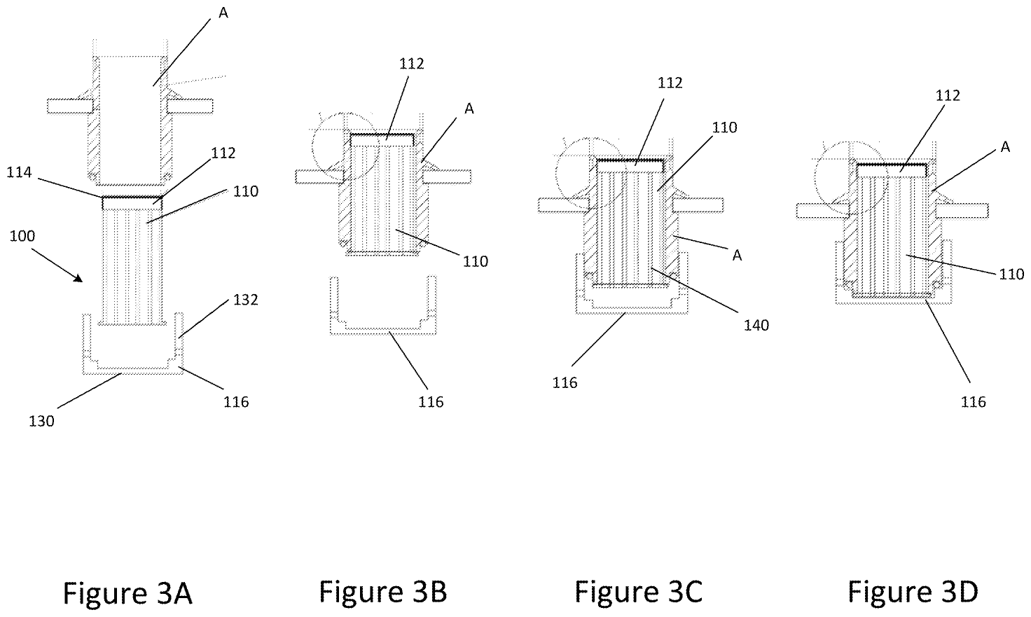

FIGS. 3A-D illustrate an example process of inserting an occlusion apparatus 100 into an inflow conduit A of a VAD. As shown in FIG. 3A, the plug tip 112 and plug surface 114 are pre-attached to the plug body 110 before it is inserted into the inflow conduit A. The plug tip 112 and plug surface 114 may be fixed to the plug body 110, or the plug tip 112 and plug surface 114 may be removeably connected to the plug body 110. If the plug tip 112 is removeably connected, the plug tip 112 is connected to the plug body 110 before implantation. The cap 116 is not attached to the plug body 110 before the plug body 110 is inserted into the inflow conduit A.

FIG. 3B shows the plug body 110 inserted within the inflow conduit A. After the motor and tubing of the VAD has been removed, the plug body 110 is inserted within the interior of the inflow conduit A. In a first embodiment, the plug body 110 fits snugly within the inflow conduit A, and there is no space between the exterior wall of the plug body 110 and the interior wall of the inflow conduit A. In another embodiment, after the plug body 110 has been inserted into the inflow conduit A, the plug body 110 is expanded to fill the space between the interior wall of the inflow conduit A and the exterior wall of the plug body 110.

At FIG. 3C, the plug cap 116 is partially inserted around the plug body 110 and the inflow conduit A. As the plug cap 116 is being attached to the plug body 110, air is allowed to escape the space between the plug cap 116 and the plug body 110. The air escapes through apertures (not shown) in the cap 116. The plug body 110 includes a plurality of grooves 140 that allow air to escape from between the exterior surface of the plug body 110 and the interior of the inflow conduit A while the plug body 110 is being inserted into the inflow conduit A.

At FIG. 3D, the plug cap 116 is secured to the plug body 110. The securement mechanism may be a screw-type attachment, a snap-fit, a friction-fit, or other type of securement mechanism known in the art. The securement mechanism may also be a quick release mechanism that allows for a removable connection to the VAD. The plug cap 116 also includes a sealing mechanism, such as an O-ring that helps the plug cap 116 to seal to the plug body 110 and inflow conduit A. The O-ring also prevents blood leakage without requiring a sealant. In an alternative embodiment, the plug cap 116 may include a hemostatic sealant instead of or in addition to an O-ring.

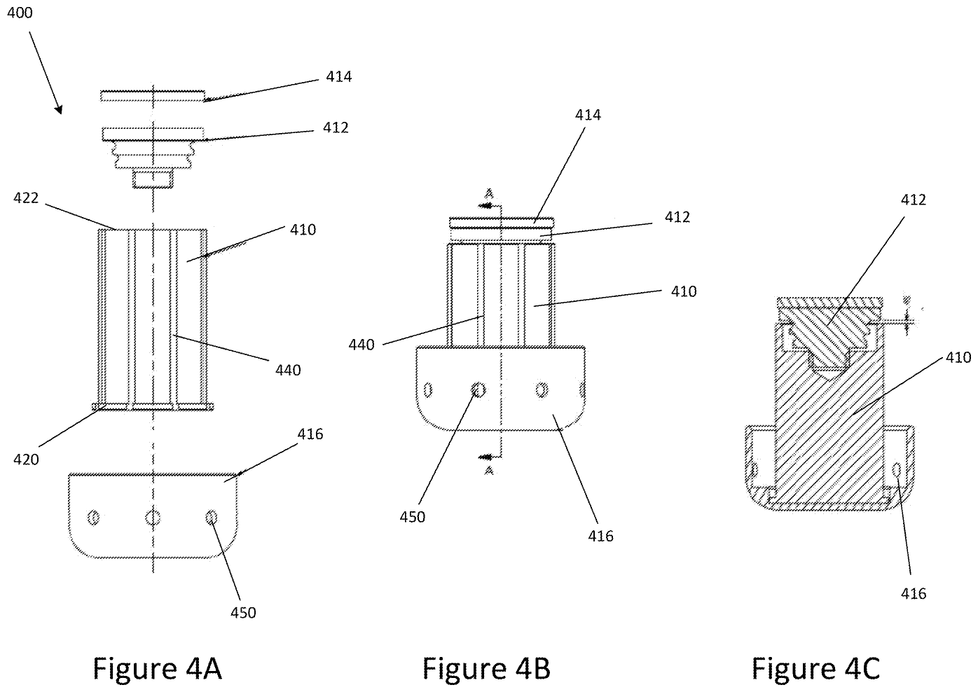

FIGS. 4A-4C illustrate an alternative embodiment of an occlusion apparatus 400. The occlusion apparatus 400 includes a plug tip 412, a plug surface 414, a plug body 410, and a plug cap 416. The occlusion apparatus 400 shown in FIG. 4A is in a disassembled state. The plug tip 412 includes a flat plug surface 414 on a superior end. The plug surface 414 includes a membrane that is in contact with blood within the heart. The membrane is made from a biocompatible material that promotes tissue ingrowth. Example materials include woven or knitted fabrics, such as DeBakey Double Velour Fabric.

In an embodiment, the plug surface 414 is removeably attached to the plug body 410 and includes an attachment mechanism (not shown) that allows it to be attached to the plug body 410. Examples of attachment mechanisms are snap-fit, screw fit, friction fit, or other similar mechanisms known in the art. In another embodiment, the plug surface 414 is fixedly connected to the plug body 410. In yet another embodiment, the plug surface 414 may be integrally formed as part of the plug body 410.

The plug body 410 is cylindrical in shape and may or may not include a lumen.

In a first embodiment, the plug body 410 includes a lumen that extends from a first end 420 to a second end 422. In another embodiment, the plug body 410 does not include a lumen, so the plug body 410 is solid, or partially filled.

The exterior surface of the plug body 410 includes a plurality of grooves 440 extending lengthwise from the first end 420 to the second end 422. In an embodiment, the plug body 410 includes 4-12 grooves. In an example embodiment, the plug body 410 has eight grooves that are equidistantly spaced around the exterior surface. The grooves 440 may be from about 1 mm to about 4 mm deep and from about 1 mm to about 4 mm wide. In an embodiment, the plug body 410 includes grooves 440 that are about 2 mm deep. In an embodiment, the plug body 410 includes grooves 440 that are about 2 mm wide. The grooves 440 function to create a channel between the left ventricle and the exterior of the heart for easy air removal during implantation.

The plug cap 416 is sized to fit over the external surface of the plug body 410 at a first end 420. The plug cap 416 has an internal diameter that is sized to fit around the external diameter of the plug body 410. The plug cap 416 also includes apertures 450, which are sized to allow air to escape through the apertures 450. For example, the apertures 450 may be about 1 mm in diameter. Alternatively, the release apertures may be less than 1 mm in diameter, for example, 0.5 mm or 0.75 mm in diameter. Still further, the apertures 450 may have a diameter of from about 1 mm to about 3 mm, for example, about 2 mm in diameter. The apertures 450 are placed every 30°-60°, for example, every 45° around the plug cap 416. As shown, the plug cap 416 includes a rounded exterior surface, although other exterior surfaces are contemplated.

In an embodiment, the plug cap 416 also includes an attachment mechanism (not shown) that allows it to be attached to the plug body 410. Examples of attachment mechanisms are snap-fit, screw fit, friction fit, or other similar mechanisms known in the art.

FIG. 4B shows the occlusion apparatus 400 in an assembled state. The plug surface 414 is attached to the plug tip 412, which is attached to a superior end of the plug body 410. In a first embodiment, the plug tip 412 is integral to the plug body 410.

In another embodiment, the plug tip 412 is fixedly attached to the plug body 410. The plug cap 416 is removeably attached to the first end 420 (or the inferior end) of the plug body 410.

FIG. 4C illustrates a cross-sectional view of the assembled state of the occlusion apparatus 400. As shown, the plug tip 412 extends within the plug body 410. The plug cap 416 extends around the external surface of the plug body 410 and creates a secure attachment.

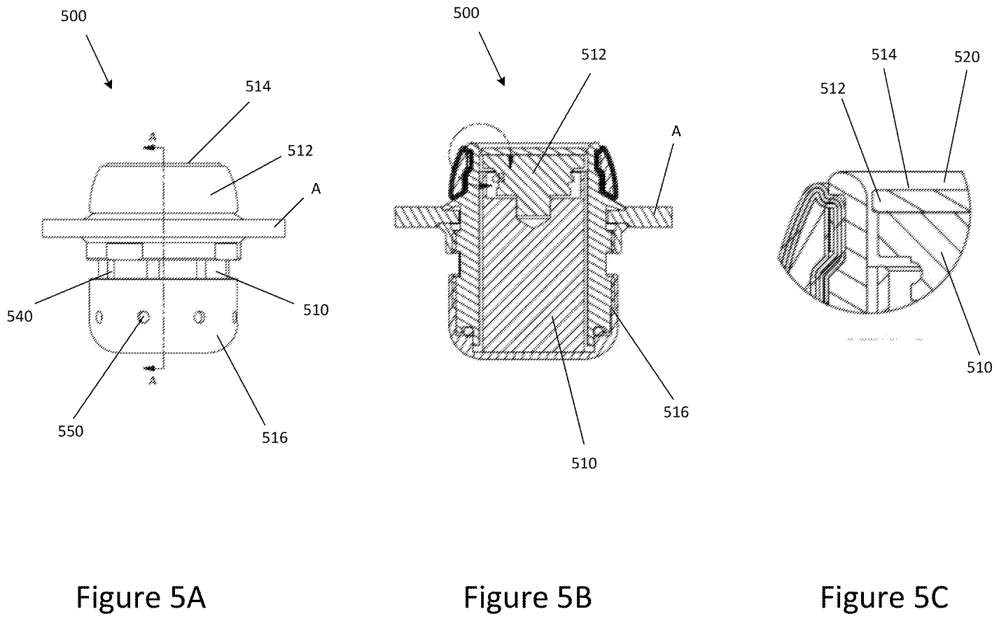

FIGS. 5A-5C illustrate another example embodiment of an occlusion apparatus 500. FIG. 5A illustrates an assembled view of the occlusion apparatus 500 including a plug tip 512 comprising a plug surface 514, a plug body 510, and a cap 516. The plug body 510 includes a plurality of grooves 540 on an external surface that extend from a superior end to an inferior end. The plurality of grooves 540 are as described above with regard to the plurality of grooves 440, the description of which is omitted for brevity.

The cap 516 includes at least one aperture 550. The at least one aperture 550 is sized to allow air to escape while the cap 516 is being inserted around the plug body 510. The at least one aperture 550 may have a diameter of from about 1 mm to about 3 mm, for example, about 2 mm in diameter, and are placed every 30°-60°, for example, every 45° around the cap 516.

FIG. 5B illustrates a cross-sectional view of the occlusion apparatus 500 along line A of FIG. 5A. In an embodiment, the plug body 510 is at least partially solid, but includes a recess at a superior end, where the plug tip 512 extends. The plug body 510 also includes a PTFE circle cushion 520 on the plug surface 514. The cushion 520 comprises polyester double velour fabric that covers the end of the plug body 510.

FIG. 5C shows a cross-sectional view of the plug body 510 and the plug tip 512. The plug body 510 includes the PTFE circle cushion 520 on the plug surface 514.

FIG. 6A illustrates a top cross-sectional view of an example embodiment of a plug body 410. The plug body 410 includes a plurality of grooves 440 arranged around the external surface of the plug body 410. As shown, the plug body 410 includes eight grooves 440. However, other numbers of grooves 440 are envisioned, for example, from 4-12 grooves 440. The plurality of grooves 440 are arranged equidistant around the surface; however, the plurality of grooves 440 may be arranged in other patterns. In an embodiment, the grooves 440 are placed every 30°-60°, for example, every 45°.

FIG. 6B shows a cross-sectional side view of the plug cap 416. The plug cap 416 includes at least one aperture 450. In an embodiment comprising more than one aperture 450, the plurality of apertures 450 are arranged equidistant around the surface; however, the apertures 450 may be arranged in other patterns. In an embodiment, the apertures 450 are placed every 30°-60°, for example, every 45°. The at least one aperture 450 may be circular in shape and have a diameter of from about 1 mm to about 3 mm, for example, about 2 mm in diameter. However, other shapes, such as ovals, squares, or triangles are envisioned.

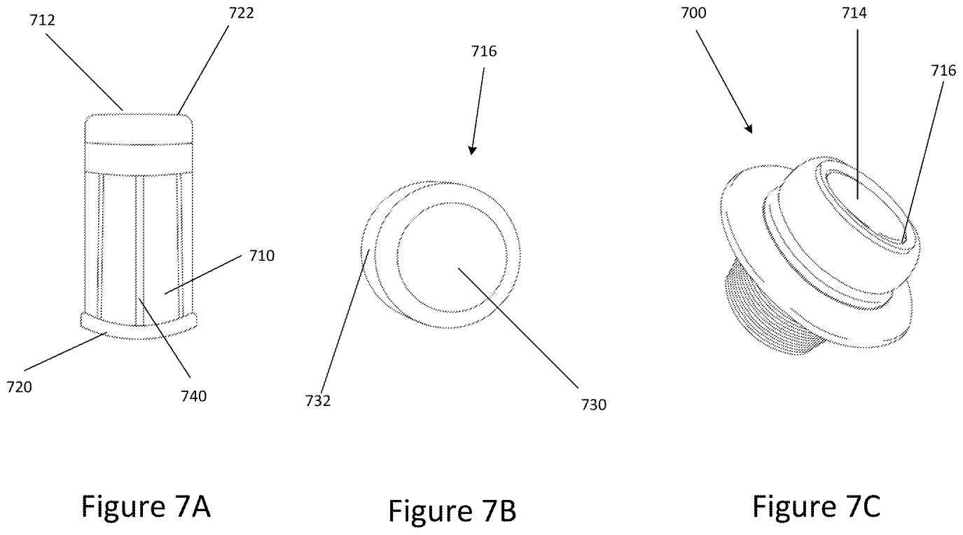

FIG. 7A shows an alternative embodiment of a plug body 710. The plug body 710 is cylindrical in shape and has a constant diameter from a superior end 722 to an interior end 720. For example, the diameter may be from about 14 mm to about 18 mm, for example, about 16 mm. The plug body 710 may be made from a biocompatible plastic, such as polypropylene, stainless steel, or titanium. A plug tip 712 is located on a superior end of the plug body 710. The plug tip 712 includes a plug surface, which is made from a material that encourages tissue ingrowth.

FIG. 7B shows an embodiment of a plug cap 716. The plug cap 716 is also cylindrical in shape, and in an embodiment, is sized to have the same inner diameter as the outer diameter of the plug body 710. In another embodiment, the plug cap 716 has a slightly smaller diameter than the plug body 710, for example, about 1 mm smaller. When covered with a fabric, the plug cap 716 has the same diameter as the plug body 710. The plug cap 716 has a diameter of from about 12 mm to about 16 mm, for example, about 15 mm.

The plug cap 716, as shown, is removable from the plug body 710, and may be attached to the plug body 710 after the plug body 710 is implanted within a patient. The plug cap 716 includes at least one aperture 750, which allows air to escape when the plug cap 716 is being attached to the plug body 710.

The cap 716 also includes a wall 732 that extends upward from the inferior surface 730. The wall 732 is located around an outside edge of the inferior surface 730 and is sized to extend around the exterior surface of the plug body 710

The plug body 710 may be made from materials selected from biocompatible plastic, such as polypropylene, stainless steel, or titanium. The plug body 710 also includes a fabric cover, made from a woven or knit fabric. The fabric cover promotes tissue ingrowth.

FIG. 7C shows an occlusion device 700 within an inflow conduit A. The plug tip 712 including the plug surface 714 extends through the inflow conduit A. The plug surface 714 is comprised of a fabric, such as a woven or knit fabric, that promotes tissue ingrowth.

As should be appreciated, the various aspects (e.g., portions, components, etc.) described with respect to the figures herein are not intended to limit the systems and methods to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

1. An apparatus for occluding an inflow conduit of a ventricular assist device, comprising:
a plug body having a superior end and an inferior end, and a cylindrical body extending from the superior end to the inferior end;
a plug surface at the superior end of the plug body, the plug surface covering the cylindrical body and having a first side and a second side; and
a plug cap configured to extend around an external surface of the cylindrical body of the plug body.

2. The apparatus of claim 1, wherein the cylindrical body of the plug body comprises an external wall having at least one groove extending from the superior end to the inferior end.

3. The apparatus of claim 2, wherein the at least one groove has a depth of from about 1 mm to about 4 mm.

4. The apparatus of claim 2, wherein the at least one grooves are spaced equidistantly around the plug body.

5. The apparatus of claim 4, wherein the at least one grooves are spaced 45° around the plug body.

6. The apparatus of claim 1, wherein the plug cap comprises at least one aperture, the aperture located on a wall of the plug cap.

7. The apparatus of claim 6, wherein the at least one aperture has a diameter of from about 0.5 mm to about 3 mm.

8. The apparatus of claim 1, wherein the first side of the plug surface comprises a fabric configured to promote tissue ingrowth, the fabric selected from textile surgical meshes and woven fabrics.

9. The apparatus of claim 1, wherein the plug body is configured to fill an internal space of the inflow conduit of the ventricular assist device.

10. The apparatus of claim 1, wherein the diameter of the plug body is from about to about 14 mm to about 18 mm.

11. The apparatus of claim 1, wherein the length of the plug body is from about 6 mm to about 12 mm.

12. The apparatus of claim 1, wherein the plug cap is removeably connected to the plug body.

13. A method for occluding a hole in the apex of the heart following an explantation procedure of a ventricular assist device, the method comprising:
inserting an occlusion apparatus into an inflow conduit of the VAD, the occlusion apparatus comprising:
a plug body having a superior end and an inferior end, a cylindrical body extending from the superior end to the inferior end, and a plug surface at the superior end of the plug body, the plug surface covering the cylindrical body and having a first side and a second side; and a plug cap located at the inferior end of the plug body.

14. The method of claim 13, wherein the plug body comprises a quick release mechanism that is removeably attached to the inflow conduit of the VAD.

15. The method of claim 13, wherein the plug surface is securely attached to the plug body before implantation into the heart.

16. The method of claim 13, wherein the method further includes attaching the plug cap to the inferior end of the plug body after the plug body is inserted within the inflow conduit.

17. The method of claim 13, wherein after the plug body is inserted in the inflow conduit the method includes expanding the plug body to fill the volume of the inflow conduit.

18. The method of claim 13, wherein the plug cap is removeably connected to the plug body.
